(19) United States
(12) Patent Application Publication
(10) Pub. No.: US 2026/0294247 A1
(43) Pub. Date: Oct. 1, 2026

(54) ACTUATION OF AN INTERNALIZED DEVICE VIA IN-BODY COMMUNICATION

(71) Applicant: Georgia Tech Research Corporation, Atlanta, GA (US)

(72) Inventors: Alex Abramson, Atlanta, GA (US); Ramy Ghanim, Atlanta, GA (US)

(21) Appl. No.: 19/571,993

(22) Filed: Mar. 19, 2026

Related U.S. Application Data
(60) Provisional application No. 63/774,377, filed on Mar. 19, 2025.

(51) Int. Cl. A61B 5/00; A61B 5/262

(52) U.S. Cl. CPC A61B 5/0028; A61B 5/262

(57) ABSTRACT

The present disclosure relates to a communication method for actuating a body-internalized device upon signal transmission; a needle-based electrode for transmitting signals throughout the body; and a thin planar electrode for receiving signals throughout the body.

### RELATED APPLICATIONS

This application claims the benefit of priority to U.S. Provisional Application No. 63/774,377, filed Mar. 19, 2025.

### GOVERNMENT SUPPORT

This invention was made with government support under grant number GM150689, awarded by the National Institutes of Health. The government has certain rights in the invention.

### BACKGROUND

Ingestible and implantable bioelectronic therapeutics often require communication modules to transfer sensing and actuation signals. Typical devices utilize Bluetooth low energy (BLE) system on chips (SoCs) or radio frequency (RF) antennas to transmit and receive these signals. However, these components are associated with significant power consumption and a locational dependence between communicating devices. Additionally, these components often increase the physical size of the bioelectronic device making them incompatible for some anatomical locations. Here we demonstrate proof of the concept of a communication system with the capability of actuating a network of internalized devices remotely from outside of the body by sending electrical pulses through the tissue. We demonstrate in vivo efficacy of the communication system in a small animal via remote stimulation of a motor nerve.

Body-conformal sensors and tissue-interfacing robotic therapeutics enable the real-time monitoring and treatment of diabetes, wound healing, and other critical conditions. By integrating sensors and drug-delivery devices, scientists and engineers have developed closed-loop drug-delivery systems with on-demand therapeutic capabilities to provide just-in-time treatments that correspond to chemical, electrical, and physical signals of a target morbidity. One of the most significant challenges is enabling these devices to communicate without exceeding device power and space constraints. A communication system entails hardware including supporting electronics and a medium for relaying signals.

Despite the demonstration of dozens of bioelectronic closed-loop therapeutic devices in small and large animal models, very few have made it into the clinic. Some have employed BLE which (1) suffers from heavy power consumption, decreasing device lifetime and (2) poor deep tissue transmission due to scattering effects. Others have used RF antennas which are less space and energy intensive-but require large external antennas and spatial alignment between transmitting and receiving antennas. These communication protocols are not adequate for developing closed-loop therapeutic systems that integrate body-worn sensors with internalized therapeutic devices such as neurostimulators and drug delivery devices. For these and other reasons, needs exist for improved in-body communication systems, devices, and methods. In our invention summary, we detail how our communication system increases device lifetime, minimizes space dedicated for hardware and supporting electronics, and decreases the need for spatial alignment between communicating devices.

### SUMMARY OF THE INVENTION

In certain aspects, the present disclosure provides a medical device system, comprising: a first electronic device in contact with tissue; a second electronic device in contact with tissue; and a pad; wherein: the first electronic device is placed inside or outside of the body; the second electronic device is placed inside or outside of the body; the first electronic device is configured to transmit an electrical signal through the tissue to the second electronic device, wherein a voltage gradient within the body is generated; and the pad is configured to harvest the generated voltage gradient, wherein the pad is placed at a predetermined distance within the body.

In certain aspects, the present disclosure provides a medical device system, comprising: a first electronic device in contact with tissue; a second electronic device in contact with tissue; and a pad; wherein: the first electronic device is placed outside of the body; the second electronic device is placed inside of the body; the first electronic device is configured to transmit an electrical signal through the tissue to the second electronic device, wherein a voltage gradient within the body is generated; and the pad is configured to harvest the generated voltage gradient, wherein the pad is placed at a predetermined distance within the body.

### BRIEF DESCRIPTION OF THE DRAWINGS

FIG. 1 In-body communication enables low-power networks of wearable and implantable therapeutics. FIG. 1A A wearable hub outfitted with a pulse generator creates a voltage gradient across tissue. Upon detection of the voltage gradient, the implantable network changes the bioelectronic's on-off state. Feedback control is accomplished by integration of a physical sensor to the wearable hub. FIG. 1B Theoretical (bars) and experimental (points, Avg±SD, n=3) power characteristics of implanted nerve-interfaced bioelectronics controlled via in-body communication (IBC), Near Field Communication (NFC), and Bluetooth Low Energy (BLE). FIG. 1C Heat map of generated voltage potentials upon application of [6V, 1 Hz, 200 μs] pulses across two hypodermic needles (gray circles) in ex vivo chicken breast models. FIG. 1D Voltage measured across a transistor's gate when applying increasing amplitude pulses across ex vivo chicken breast tissue. Shaded regions denote transistor gates and threshold voltages. (black line=avg, grey line=individual tests; n=9 across 3 chicken breasts) FIG. 1E Voltage measured across each transistor's gate when moving the harvesting probes away from the signal injection source. Measurements taken at different pulse amplitudes. (line=Avg±SD, n=9 across 3 chicken breasts)

FIG. 2 Electrode surface area. Probes can have as low as 3.14 mm 2 surface area and still detect adequate voltage gradients in tissue. (line=Avg, n=9 across 3 chicken breasts; shaded region=SD).

FIG. 3 In-body communication architecture. A custom flexible printed circuit board (FPCB) continuously monitors a strain sensor and automatically emits a variable (1-12 V) square wave pulse to trigger the implantable network. Bioelectronics can be switched individually or synergistically.

FIG. 4 Microneedle Lead Architecture. FIG. 4A Photo of stainless-steel microneedles (inset scale bar=1mm). FIG. 4B Schematic of skin-penetrating microneedles in in-body communication architecture. FIG. 4C Representative pulses of transmitting [4V, 1 Hz, 1 ms] square wave pulses using a skin-interfaced electrode and microneedles in vivo in rats. FIG. 4D Peak injection current across microneedles in vivo in rats (n=18 across 3 rats, SD plotted).

FIG. 5 In Vivo Voltage Gradient Harvesting and Tissue Response in Various Anatomical Locations FIGS. 5A-5C In vivo voltage gradient harvesting in the subcutaneous space FIG. 5A, intraperitoneal space FIG. 5B, and the stomach FIG. 5C (gray lines=individual trials, black line=average). FIG. 5D In vivo voltage gradient harvesting in the subcutaneous space as microneedles are placed in variable orientations across a small animal (n=5 in 1 rat). FIG. 5E Representative tissue histology images illustrating no electrical stimulation, moderate levels of electrical stimulation, and high levels of electrical stimulation.

FIG. 6 Possible ways of implementation of the medical device system in the Human.

FIG. 7 The size comparison between the communication system disclosed herein and other communication systems. Clockwise from the bottom right, the systems shown are a Bluetooth system on chip, a millimeter-scale working distance NFC antenna, and a centimeter-scale working distance NFC antenna. At the bottom, the communication system disclosed herein is depicted inside of a 16-gauge needle.

FIG. 8 Continued Ex Vivo characterization of in-body communication and transistor switching. FIG. 8A Harvesting pads are maintained at a constant distance of 3 cm, while the signal injection pad gets closer to the harvesting pad. (Black lines=mean, n=3). FIG. 8B Various harvesting pad surface areas at a constant applied voltage of 7V and separation of 3 cm between harvesting pads. FIG. 8C Transfer curve for P1-50 OMEIC OECT device. FIG. 8D Application of 10 1 s pulses [0,−0.8V, 0.5 Hz], V_{ds}=−0.6V. FIG. 8E Application of 10 100 ms pulses [0,−0.8V,0.5 Hz], V_{ds}−0.6V. FIG. 8F OECT used to switch on/off an LED using an ex vivo chicken breast model.

FIG. 9 Integrating various actuators for communication via IBC ex vivo. FIG. 9A Ex vivo set up for switching on various electromechanical devices including an LED and a pump. The gate pins of the transistor are connected to tissue-interfacing harvesting probes. The electromechanical device is illustrated as a resistive load in the schematic. FIG. 9B Utilizing a PNP and an NPN along with a [10V,0V,1 s] pulse allows for selective triggering of 2 LEDs. FIG. 9C Utilizing a PNP and a MOSFET along with a [6.5V,0V,3 s] pulse allows for selective triggering of 2 pumps.

FIG. 10 Device variations for multiplexed control. FIG. 10A Adding a resistor between the harvesting pad and NPN transistor base (NPN-R) ex vivo in chicken breast increases the required applied voltage for switching (black=avg, grey=ind; n=10). FIG. 10B Adding a capacitor between the base and emitter of an NPN transistor (NPN-C) ex vivo in chicken breast increases the required applied pulse length for switching (Avg 540±SD; n=6-11). FIG. 10C Low voltage long pulses only trigger NPN-C switches ex vivo in chicken breast while high voltage short pulses only trigger NPN-R switches. (R=2.2 kΩ2; C=1 μF; ind points, Avg ±SD, n=8). FIG. 10D Heat map of generated voltage potentials upon application of [10 V, 1 Hz, 200 μs] pulses using hypodermic needles [gray circles] in ex vivo skin-on, bone in, pork belly (n=3). FIG. 10E Voltage measured across each transistor's gate at different depths in ex vivo pork 545 belly. (line =Avg ±SD, n=9).

FIG. 11 Microneedle current injection stability. Peak injection current across various skin-interfaced electrodes in vivo in rats (n=18 across 3 rats, SD plotted).

FIG. 12 Working areas permitted by various communication modalities estimated by key performance metrics associated with each modality. FIG. 12A Inductive coupling between TX and RX antennas. FIG. 12B NFC link between TX and RX antennas. FIG. 12C BLE link between TX and RX system on chips. FIG. 12D Smart Wireless Autonomous Networking System (SWANS) link between TX and RX implants. All scale bars are 5 cm.

FIG. 13 Smart Wireless Autonomous Networking System (SWANS) inter-implant communication. FIG. 13A Implant relay where an NPN implant [white] and NPN-R implant [blue] are connected via in-body ionic communication. The wearable can emit a low amplitude pulse and trigger only the white implant which then triggers the higher threshold blue implant through a higher magnitude pulse. FIG. 13B Current through blue implant during wearable application of low and high amplitude pulses in the presence and absence of the white implant (n=30 per group). FIG. 13C Triggering of the blue NPN-R implant is conditional and can be a function of a sensed variable such as temperature by utilizing a thermistor in NPN-R control circuit. FIG. 13D Current through the actuating blue implant in the presence of the white implant during application of low amplitude pulses at T<40° C. and at T>40° C. (n=30 per group).

FIG. 14 Current detected by wearable device following implant actuation allows for actuation confirmation. When the implant emits a pulse into the surrounding tissue after actuating, this pulse can be detected by the microneedles in the wearable device, confirming implant actuation. Here we demonstrate the detected current by the wearable after a 3V, 1.5V, or no pulse [as a negative control] emitted by the implant in an ex vivo swine pork belly model (n=30). The blue region encompasses current levels associated with active pulsing by the implant.

FIG. 15 Smart Wireless Autonomous Networking System (SWANS) network is enabled by numerous transistors, each with its characteristic behavior allowing for selective and combinatorial activation of multiple devices.

FIG. 16 Comparator circuits allow for selective triggering based on voltage gradient windows. FIG. 16A Comparator circuit diagram. FIG. 16B Image of three SWANS comparator circuit implants, one NPN transistor only circuit implant, and a US quarter. FIG. 16C Applying increasing voltages allows for selective control over 3 or more devices. Based on this ex-vivo testing, we recommend that voltage windows below 0.1V are avoided. As pulses of increasing voltages were tested on the tissue, we observed an imperfect drop off in the falling edge of the harvested voltage response. This is likely an effect of the capacitive function of tissue, which can store some charge, delaying the voltage drop. As voltages increased, the drop off voltage approached 0.1V. As a result, the voltage output of implant C moderately increased, though remained low enough to be at an off state. FIG. 16D Representative output voltages from comparator circuit upon application of 0.1V, 0.3V, and 0.5V across the tissue-interfacing receiver pads. FIG. 16E Power characteristics of NPN transistor only circuit compared to comparator. (Bars=theoretical estimate, Data point=experimental measurement, Avg±SD, n=3 devices) All scale bars=1 cm.

FIG. 17 Images of ex vivo pork tissue for characterizing generated electric fields. FIG. 17 (A) Skin on pork belly cross-section including skin, fat, muscle, bone, and viscera. FIG. 17B Skin-on pork belly for decimeter-scale electric field generation and voltage gradient harvesting.

FIG. 18 Smart Wireless Autonomous Networking System (SWANS) NPN implants do not trigger differently in the presence of a static discharge. NPN implants turned on in the presence of a 4 V SWANS stimulation both in the presence and absences of a static discharge. NPN implants did not turn on when a static discharge along with a 1 V SWANS stimulation were applied to the tissue (n=30 triggers, Avg.±SD).

FIG. 19 Smart Wireless Autonomous Networking System (SWANS) microneedle 10-day implant histology. (Top) H&E-stained non microneedle treated skin samples and microneedle-treated skin samples after 10 days of microneedle implantation without electrical stimulation. (Bottom) Masson's Trichrome stained non-microneedle treated skin samples and microneedle-treated skin samples after 10 days of microneedle implantation without electrical stimulation. Scar tissue formed above the stratum corneum after implantation of microneedles (brown in Masson's Trichrome). All scale bars=100 μm.

FIG. 20 Chronic Smart Wireless Autonomous Networking System (SWANS) use for two months. FIG. 20A Experimental outline for chronic SWANS deployment. FIG. 20B Tuning procedure on week 0 and week 8 illustrating that TX-RX link can be restored by increasing the applied voltage following a failed actuation event [red] until a successful actuation event [green] occurs. FIG. 20C Implant current measured over time when triggered with SWANS wearable transmitter (n=30 triggers per recording session, Avg±SD). Applied voltages in each rat are increased over time relative to the initially delivered voltage pulse to ensure successful actuation events. FIG. 20D Rat weight over time during chronic SWANS use All rats showed no signs of distress during the study, and they all survived until their pre-prescribed euthanasia timepoints [black dots].

FIG. 21 Smart Wireless Autonomous Networking System (SWANS) 2-month use skin histology. Representative histology skin tissue samples for control [C] and experimental [E] groups where control group received no treatment and experimental group underwent two months of repeat stimulation with [10 V, 2 ms, 1 Hz] pulses. Experimental skin samples include samples taken at the microneedle site and away from the microneedle site. Stains include hematoxylin and eosin (H&E), cleaved caspase 3 (CC3), antigen Kiel 67 (ki67), and 4-Hydroxynonenal (4-HNE). All scales bars=100 μm.

FIG. 22 Smart Wireless Autonomous Networking System (SWANS) 2-month use internal organ histology. Representative histology of off target tissue samples for control and experimental groups where control group received no treatment and experimental group underwent two months of repeat stimulation with [10 V, 2 ms, 692 1 Hz] pulses. Stains include hematoxylin and eosin (H&E), cleaved caspase 3 (CC3), ki67, and 4-Hydroxynonenal (4-HNE). All scale bars=100 μm.

FIG. 23 Electrocardiograms following SWANS stimulation. FIG. 23A Representative ECG pattern during pulse [highlighted] and during rest periods [not highlighted]. FIG. 23B The duration between the S and T waves in a rat ECG with a naïve rat under anesthesia's ST period highlighted in gray (n=30 pulses across 3 animals, Avg.±SE). FIG. 23C The % difference in R peak amplitude from baseline (n=30 pulses across 3 animals, Avg.±SD).

FIG. 24 Peripheral nerve recordings following SWANS Stimulation. FIG. 24A Direct stimulation of sciatic nerve with pulse generator. FIG. 24B Sciatic nerve recording during whole body variable amplitude pulse application without SWANS receiver or associated nerve cuff. FIG. 24C Sciatic nerve recording during whole body pulse application with SWANS receiver and nerve cuff. FIG. 24D Sciatic nerve recording without whole body pulse application with SWANS receiver and nerve cuff. FIG. 24E Vagus nerve recording during whole body pulse application without SWANS receiver or associated nerve cuff (n=10 pulses, all lines drawn individually).

### DETAILED DESCRIPTION OF THE INVENTION

In certain aspects, the present disclosure provides a medical device system, comprising: a first electronic device in contact with tissue; a second electronic device in contact with tissue; and a pad; wherein: the first electronic device is placed inside or outside of the body; the second electronic device is placed inside or outside of the body; the first electronic device is configured to transmit an electrical signal through the tissue to the second electronic device, wherein a voltage gradient within the body is generated; and the pad is configured to harvest the generated voltage gradient, wherein the pad is placed at a predetermined distance within the body.

In certain aspects, the present disclosure provides a medical device system, comprising: a first electronic device in contact with tissue; a second electronic device in contact with tissue; and a pad; wherein: the first electronic device is placed outside of the body; the second electronic device is placed inside of the body; the first electronic device is configured to transmit an electrical signal through the tissue to the second electronic device, wherein a voltage gradient within the body is generated; and the pad is configured to harvest the generated voltage gradient, wherein the pad is placed at a predetermined distance within the body.

In certain aspects, the present disclosure provides for passage of an electrical signal from an electronic device touching tissue to another device touching tissue through the tissue in order to generate a voltage gradient in the body that can be harvested by a pad placed at a given distance in the body.

In certain embodiments, the medical device comprises: a wearable printed circuit board (PCB); an implantable printed circuit board (PCB); and a harvesting pad; wherein: the wearable PCB is configured to transmit an electrical signal to the implantable PCB through the tissue, wherein a voltage gradient within the body is generated; and the harvesting pad is configured to harvest the generated voltage gradient, wherein the harvesting pad is placed at a predetermined distance within the body.

In certain embodiments, the implantable PCB further comprises one or more transistors, wherein the transistor(s) is/are switched on and off by the harvested voltage gradient.

In certain embodiments, one or more of the transistors is/are an NPN transistor, and the harvesting pads are connected to the base and emitter pins of the NPN transistor. In further embodiments, the NPN transistor is switched on by a harvested voltage gradient of approximately 0.7 V.

In other embodiments, one or more of the transistors is/are a PNP transistor, and the harvesting pads are connected to the base and collector pins of the PNP transistor. In further embodiments, the PNP transistor is switched on by the harvested voltage gradient of approximately 0.1 V.

In certain embodiments, the transistor(s) is/are placed in series and/or parallel with other passive electronic components selected from other transistors, capacitors, resistors, and/or inductors.

In certain embodiments, one or more of the transistors is/are a MOSFET or FET transistor, and the harvesting pads are connected to the gate and source pins of the MOSFET or FET transistor. In further embodiments, the transistor(s) is/are a P55NF06L MOSFET transistor, wherein the P55NF06L MOSFET transistor is switched on by a harvested voltage gradient of approximately 0.5 V. In alternative embodiments, the transistor(s) is/are a MOSFET transistor other than a P55NF06L MOSFET transistor, and wherein the transistors have differing requirements for harvested voltage gradients.

In certain embodiments, one or more of the transistor(s) is/are an organic electrochemical transistor (OECT), and the harvesting pads are connected to the gate and source pins of the OECT transistor. In further embodiments, the OECT is switched on by a harvested voltage gradient of approximately 0.8 V. In yet further embodiments, the OECT is kept on for a longer period of time by a series of pulses applied to an OECT transistor. In additional embodiments, the OECT is kept on for a longer period of time by a longer pulse applied to an OECT transistor.

In certain embodiments, the implantable PCB further comprises a memristor, wherein the harvesting pads are connected to the memristor.

In certain embodiments, the device comprises more than one transistors, wherein the transistors receive the signals emitted by the harvesting probes independently.

In certain embodiments, one or more of the transistors is/are a PNP or a MOSFET, wherein the PNP or MOSFET can be independently triggered based on the voltage pulse emitted.

In certain embodiments, one or more of the transistors is/are a PNP or an NPN, wherein the PNP or NPN can be independently triggered based on the voltage pulse emitted.

In certain embodiments, one or more of the transistors is/are a MOSFET, wherein the MOSFET can be independently triggered; and/or the transistors are a MOSFET and an NPN, wherein the MOSFET and NPN can be triggered together based on the voltage pulse emitted.

In certain embodiments, one or more of the transistors is/are selected from an OECT, NPN or MOSFET, or PNP, wherein the OECT, NPN or MOSFET, and PNP can be independently triggered based on the voltage pulse, pulse length, and number of pulses emitted. In certain embodiments, the transistors are a MOSFET and an NPN, wherein the MOSFET and NPN can both be added and triggered based on the voltage pulse, pulse length, and number of pulses emitted.

In certain embodiments, the device comprises two transistors, wherein the transistors are on different devices placed in different locations in the body with different harvesting probes or harvesting pads. In alternative embodiments, the device comprises two transistors, wherein the transistors are on different devices placed in the same location in the body with different harvesting probes. In other embodiments, the device comprises two transistors, wherein the transistors are on the same device placed in the same location in the body with different harvesting probes. In yet further alternative embodiments, the device comprises two transistors, wherein the transistors are on the same devices placed in the same location in the body with the same harvesting probes.

In certain embodiments, the voltage gradient is generated by applying the pulse to the epidermis.

In certain embodiments, a conductive gel is used.

In certain embodiments, the wearable PCB further comprises needles, wherein the needles are configured to pre-make holes in the epidermis.

In certain embodiments, the voltage gradient is generated by applying the pulse below the epidermis.

In certain embodiments, the wearable PCB further comprises microneedles, microneedle pads, and/or microneedle patches, wherein microneedles, microneedle pads and/or microneedles patches are configured to apply the pulse below the epidermis.

In certain embodiments, the microneedles are of 250 μm or greater length to reach below the epidermis. In further embodiments, the microneedles are of 1000 μm or lesser length to reduce the pain associated with injection.

In certain embodiments, the microneedles are made from a biocompatible conductive material selected from stainless steel, gold, and PEDOT:PSS.

In certain embodiments, the microneedles do not provide greater than 5 mA of current to the subdermal tissue.

In certain embodiments, the microneedles have a surface area equal to approximately 0.75 mm². In other embodiments, the microneedles have a surface area greater than approximately 0.75 mm². In alternative embodiments, the microneedles have a surface area less than approximately 0.75 mm².

In certain embodiments, the microneedles have a surface area less than approximately 0.75 mm², and the voltage pulse needs to provide a greater minimum voltage to generate intrabody voltage gradients.

In certain embodiments, the microneedles can reach depths of approximately 0.9, 0.75, 0.6 or 0.25 mm below the epidermis.

In certain embodiments, the microneedles have 3, 4, 6, or 23 appendages.

In certain embodiments, the microneedle pads can provide a 7V minimum voltage pulse to generate a voltage gradient of 0.7 V or more. In further embodiments, the microneedle pads can provide a 3.5V minimum voltage pulse to generate a voltage gradient of 0.5 V or more. In yet further embodiments, the microneedle pads can provide a 1V minimum voltage pulse to generate a voltage gradient of 0.1 V or more.

In certain embodiments, the device comprises two microneedle patches, wherein the microneedle patches are applied to the tissue, and one acts as the input voltage and the other acts as a ground.

In certain embodiments, one microneedle patch is applied to the tissue and the Earth acts as a ground.

In certain embodiments, the device further comprises harvesting pads, wherein the harvesting pads are made of conductive or semiconductor material, and the harvesting pads are to harvest the voltage gradient inside of the body.

In certain embodiments, the cross-sectional area of the harvesting pad can be as small as 1 mm² or less.

In certain embodiments, the harvesting pads can be placed a minimum of 5 cm apart or less to harvest a voltage gradient of 0.7 V or more. In further embodiments, the harvesting pads can be placed a minimum of 3 cm apart or less to harvest a voltage gradient of 0.3 V or more. In yet further embodiments, the harvesting pads can be placed a minimum of 0.5 cm apart or less to harvest a voltage gradient of 0.1 V or more.

In certain embodiments, the harvesting pads are connected to one or more transistors.

In certain embodiments, the harvesting pads are connected to a device that directly powers, and the device is selected from the group consisting of a neurostimulator, a degradable metal film, a pump, a charger, a light emitting diode (LED), a triggerable auto-injector, a motor, a pneumatic inflater, an electrolyser, and a microcontroller.

In certain embodiments, the device further comprises a transistor, wherein the transistor is utilized to switch on a device, where the device is selected from the group consisting of a neurostimulator, a degradable metal film, a pump, a charger, a light emitting diode (LED), a triggerable auto-injector, a motor, a pneumatic inflater, an electrolyser, and a microcontroller; wherein the device is for the purposes selected from neurostimulation, drug delivery, biosignal collection such as temperature, pH, gas concentrations, oxygen generation to treat and monitor diabetes, wound healing, myocardial infarctions, epilepsy, and Parkinson's disease.

In certain embodiments, the device further comprises a wearable printed circuit board (PCB), wherein the wearable PCB produces an emitted pulse.

In certain embodiments, the wearable printed circuit board further comprises a sensor connected to the wearable PCB, wherein the sensor is a chemical or physical sensor selected from a group consisting of a glucose sensor, a physiological analyte, a strain sensor, a pressure sensor, an EEG sensor, and an EKG sensor; and the wearable PCB can emit the pulse when the sensor reaches a threshold value.

In certain embodiments, the wearable printed circuit board can emit the pulse when a user inputs a command by touching the wearable to emit the pulse.

In certain embodiments, the wearable printed circuit board can emit the pulse when a user input a command by using a smartphone app that sends a signal to the wearable printed circuit board

In certain embodiments, the wearable printed circuit board can emit the pulse at a predetermined or preprogrammed frequency and/or schedule wherein the frequency and/or schedule is selected from once every second, once every minute, once every hour, or once every day.

In certain embodiments, the frequency and/or schedule are selected from approximately once every second, once every 2 seconds, once every 3 seconds, once every 4 seconds, once every 5 seconds, once every 6 seconds, once every 7 seconds, once every 8 seconds, once every 9 seconds, once every 10 seconds, once every 11 seconds, once every 12 seconds, once every 13 seconds, once every 14 seconds, once every 15 seconds, once every 16 seconds, once every 17 seconds, once every 18 seconds, once every 19 seconds, once every 20 seconds, once every 21 seconds, once every 22 seconds, once every 23 seconds, once every 24 seconds, once every 25 seconds, once every 26 seconds, once every 27 seconds, once every 28 seconds, once every 29 seconds, once every 30 seconds, once every 31 seconds, once every 32 seconds, once every 33 seconds, once every 34 seconds, once every 35 seconds, once every 36 seconds, once every 37 seconds, once every 38 seconds, once every 39 seconds, once every 40 seconds, once every 41 seconds, once every 42 seconds, once every 43 seconds, once every 44 seconds, once every 45 seconds, once every 46 seconds, once every 47 seconds, once every 48 seconds, once every 49 seconds, once every 50 seconds, once every 51 seconds, once every 52 seconds, once every 53 seconds, once every 54 seconds, once every 55 seconds, once every 56 seconds, once every 57 seconds, once every 58 seconds, once every 59 seconds, and once every 60 seconds.

In certain embodiments, the frequency and/or schedule are selected from approximately once every minute, once every 2 minutes, once every 3 minutes, once every 4 minutes, once every 5 minutes, once every 6 minutes, once every 7 minutes, once every 8 minutes, once every 9 minutes, once every 10 minutes, once every 11 minutes, once every 12 minutes, once every 13 minutes, once every 14 minutes, once every 15 minutes, once every 16 minutes, once every 17 minutes, once every 18 minutes, once every 19 minutes, once every 20 minutes, once every 21 minutes, once every 22 minutes, once every 23 minutes, once every 24 minutes, once every 25 minutes, once every 26 minutes, once every 27 minutes, once every 28 minutes, once every 29 minutes, once every 30 minutes, once every 31 minutes, once every 32 minutes, once every 33 minutes, once every 34 minutes, once every 35 minutes, once every 36 minutes, once every 37 minutes, once every 38 minutes, once every 39 minutes, once every 40 minutes, once every 41 minutes, once every 42 minutes, once every 43 minutes, once every 44 minutes, once every 45 minutes, once every 46 minutes, once every 47 minutes, once every 48 minutes, once every 49 minutes, once every 50 minutes, once every 51 minutes, once every 52 minutes, once every 53 minutes, once every 54 minutes, once every 55 minutes, once every 56 minutes, once every 57 minutes, once every 58 minutes, once every 59 minutes, and once every 60 minutes.

In certain embodiments, the frequency and/or schedule are selected from approximately once every hour, once every 2 hours, once every 3 hours, once every 4 hours, once every 5 hours, once every 6 hours, once every 7 hours, once every 8 hours, once every 9 hours, once every 10 hours, once every 11 hours, once every 12 hours, once every 13 hours, once every 14 hours, once every 15 hours, once every 16 hours, once every 17 hours, once every 18 hours, once every 19 hours, once every 20 hours, once every 21 hours, once every 22 hours, once every 23 hours, once every 24 hours, once every 25 hours, once every 26 hours, once every 27 hours, once every 28 hours, once every 29 hours, once every 30 hours, once every 31 hours, once every 32 hours, once every 33 hours, once every 34 hours, once every 35 hours, once every 36 hours, once every 37 hours, once every 38 hours, once every 39 hours, once every 40 hours, once every 41 hours, once every 42 hours, once every 43 hours, once every 44 hours, once every 45 hours, once every 46 hours, once every 47 hours, and once every 48 hours.

In certain embodiments, the device further comprises a circuit board inside of the body, wherein the circuit board produces an emitted pulse and wherein the circuit board is implanted, ingested, or inserted.

In certain embodiments, the emitted pulse comes from the gastrointestinal (GI) tract, the subcutaneous space, the intraperitoneal space, the nasal cavity, the epidermal space, the subdermal space, the intrathoracic space, or a space within an organ or on the wall of an organ, wherein the circuit board is implanted within the organ or on the wall of the organ

In certain embodiments, the emitted pulse is logic-gated and is only emitted when a criterion is met. In some embodiments, the criterion is a temperature, pressure, chemical composition, or pH.

In certain embodiments, the device further comprises harvesting pads, wherein the harvesting pads are placed inside of the body, wherein the harvesting pads are implanted, ingested, or inserted in the body.

In certain embodiments, the harvesting pads are located in the gastrointestinal (GI) tract, the subcutaneous space, the intraperitoneal space, the nasal cavity, the epidermal space, the subdermal space, the intrathoracic space, or a space within an organ or on the wall of an organ, wherein the circuit board is implanted within the organ or on the wall of the organ.

In certain embodiments, the harvesting pads are located in different organs in the body.

In certain embodiments, the device further comprises harvesting pads, wherein the harvesting pads are placed as wearable.

In certain embodiments, the device further comprises harvesting pads and one or more transistors, wherein the harvesting pads are implantable, and the transistor(s) measures no greater than 158 mm² in surface area.

In certain embodiments, in-body communication pathway improves the battery life of receiving devices compared to devices that use Bluetooth or NFC.

In certain embodiments, the in-body communication pathway improves the battery life of emitting devices compared to devices that use Bluetooth or NFC.

In certain embodiments, the first electronic device is a first printed circuit board (PCB) and the second electronic device is a second printed circuit board (PCB).

In certain embodiments, the first circuit board produces an emitted pulse and the second circuit board receives an emitted pulse.

In certain embodiments, the emitted pulse is logic-gated and is only emitted when a criterion is met.

In certain embodiments, the received pulse is logic-gated and is only received when a criterion is met.

In certain embodiments, the first printed circuit board (PCB) further comprises a transistor and a sensor, and the sensor only allows the transistor to turn on when a criterion is met.

In certain embodiments, the second printed circuit board (PCB) further comprises a transistor and a sensor, and the sensor only allows the transistor to turn on when a criterion is met.

In certain embodiments, the criterion is a temperature, pressure, chemical composition, resistivity, or pH.

In certain embodiments, the first electronic device is an implantable printed circuit board (PCB) and the second electronic device is a wearable printed circuit board (PCB), and the implantable PCB is configured to transmit an electrical signal through the tissue to the wearable PCB.

In certain embodiments, the first electronic device is a first wearable printed circuit board (PCB) and the second electronic device is a second wearable printed circuit board (PCB), and the first wearable PCB is configured to transmit an electrical signal through the tissue to the second wearable PCB.

In certain embodiments, the first electronic device is a first implantable printed circuit board (PCB) and the second electronic device is a second implantable printed circuit board (PCB), and the first implantable PCB is configured to transmit an electrical signal through the tissue to the second implantable PCB.

In certain embodiments, the first electronic device is an implantable printed circuit board (PCB) and the second electronic device is an ingestible printed circuit board (PCB), and the implantable PCB is configured to transmit an electrical signal through the tissue to the ingestible PCB.

In certain embodiments, the first electronic device is a first ingestible printed circuit board (PCB) and the second electronic device is a second ingestible printed circuit board (PCB), and the first ingestible PCB is configured to transmit an electrical signal through the tissue to the second ingestible PCB.

In certain embodiments, the first electronic device is an ingestible printed circuit board (PCB) and the second electronic device is a wearable printed circuit board (PCB), and the ingestible PCB is configured to transmit an electrical signal through the tissue to the wearable PCB.

In certain embodiments, the first electronic device is a wearable printed circuit board (PCB) and the second electronic device is an ingestible printed circuit board (PCB), and the wearable PCB is configured to transmit an electrical signal through the tissue to the ingestible PCB.

In certain embodiments, the electrical signal is a voltage pulse.

In certain embodiments, the first electronic device produces a voltage gradient.

In certain embodiments, the first electronic device generates an electric field.

In certain embodiments, the pad is configured to harvest the generated electric field.

In certain embodiments, the pad is connected to the second electronic device.

While previous examples of in body communication have used signals passed from inside or outside of the body to another part of the body in order to denote the presence of another object, never before has an electrical pulse passed through body tissue been used to switch on or off a device inside of the body. The novelty in this proposal stands from: the ability to perform this switching action; that the switching action can be triggered based on the readout of a sensor elsewhere; characterization of the required pulses necessary to perform this switching action in multiple transistor devices; characterization of the necessary harvesting pad sizes, materials, thicknesses, and distances apart needed to harvest the voltage gradient; the characterization of passage of a pulse from the epidermis and from the dermis; the use of microneedles to deliver the pulse through the dermis; the current thresholds needed to ensure that tissue damage does not occur; the locations and orientations at which the harvesting pads can be placed in the body compared to the stimulation probes; the fact that multiple devices throughout the body can be triggered independently using the same hub but different pulses; that the device has applications in therapeutics such as neurostimulation and drug delivery.

Safety: To create a safe communication system, it is necessary to have the following: (1) a limit on the amount of current that passes through the body [currents higher than 5 mA will cause damage, and we provide histology demonstrating that there are current values that do not stimulate high levels of scar tissue formation]. (2) Microneedles are minimally invasive, and the placement of the microneedles in the skin for extended periods of time could cause fibrosis and reduce efficacy or create an infection [there is precedent to having devices inside of the subcutaneous space for up to 10 days in the continuous glucose monitor]. (3) the materials in the body must be biocompatible [the only materials that are required to be in direct contact with the body are the injection probes and the harvesting pads, and these can be made from any biocompatible conductor or semiconductor. (4) The speed at which the implantable reacts to the signal is critical to maintaining precision control. This means both the time that it takes to turn on and the time that it remains on after the signal is sent.

### EXAMPLES

### Example 1: Method for Actuating a Body-Internalized Device Upon Signal Transmission

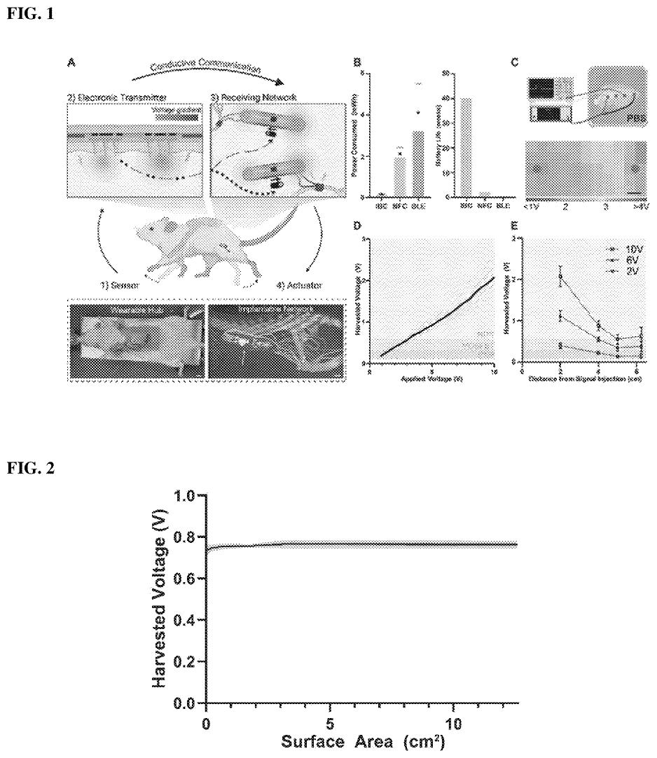

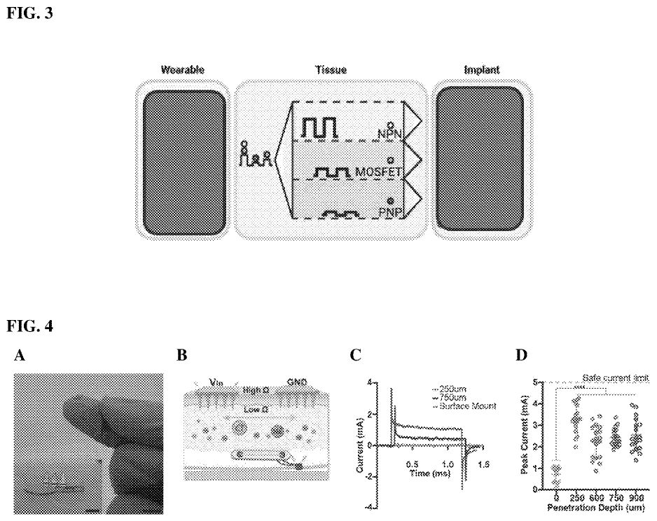

In electrolyte environments, such as saline-rich tissue, voltage gradients can be generated by applying a square wave pulse across two electrolyte interfacing electrodes, such as stainless-steel microneedles. This can be produced using different signals including different frequencies (examples ranging from 0.1 Hz to 10,000 Hz, but they can extend from this range too), different voltages (0-10V, but they can extend beyond this range too), and different pulse widths (1 μs to 5 s, but they can extend from this range as well). These signals lead to variable generated voltage gradients across the tissue. It is possible to use these voltage gradients to trigger a transistor switch, a voltage gated switch (FIG. 1A). If the detected voltage gradient is greater than the transistor's threshold, the transistor allows current to flow through the circuit, while if it is less than the threshold, current cannot flow through the circuit. The use of a transistor switch allows for regulation of a circuit's on/off state. Commercial transistors such as 2N2222 NPN, 2N2907 PNP, and P55NF06L MOSFET can be facilely integrated with this framework. Other transistors will also work. Experimental transistors such as organic electrochemical transistors can also be used.

### Example 2: Method for Actuating Multiple Body-Internalized Devices Upon Signal Transmission

A combination of transistor selection (and the transistor's characteristic threshold voltage) and transmitted electrical pulse allows for precise control over multiple dispersed devices synchronously or asynchronously. The framework described here shares a lot of traits with BLE and RF data transfer. The body-internalized devices have a transistor that is continuously in a “Standby” state where the device is continuously and/or passively reading a voltage gradient. Because the device is continuously and/or passively reading a voltage gradient, our framework allows for device to simultaneously be in “Standby” and “Advertising” state. Because of this design, the in-body communication integrated device has 15× greater power efficiency than NFC and 29× greater power efficiency than BLE (FIG. 1B). Additionally, our framework allows for a “Connection” state where the transmitter device is actively transmitting a signal, and the receiver device is actively receiving said signal. A high amplitude pulse could trigger one transistor, and a low amplitude could trigger a different transistor. A high amplitude pulse could trigger two transistors, and a low amplitude pulse could trigger a different transistor. A low amplitude pulse could trigger one transistor. A high amplitude pulse could trigger one transistor.

### Example 3: Method for Actuating a Device Upon Signal Transmission Upon Detecting a Signal from a Disease Morbidity

Signal transmission can occur due to user input (e.g., user inputs command onto smart phone which relays data to the wearable, thereby actuating a device). Signal transmission can occur on a preprogrammed frequency or schedule such as once a second, once every minute, once every hour, once every day, once every month, etc. Signal transmission can also be tied to a sensor such as a body-worn strain sensor or some other physical sensor. A chemical sensor such as a continuous glucose monitor, or a ketone monitor could also be used. Once a threshold value is crossed with regards to the sensing objective, signal transmission could automatically happen from a printed circuit board. Additionally, a control algorithm (such as a proportional, integral, and derivative (PID) controller, model predictive control (MPC) controller, or a fuzzy logic control (FLC) controller), could control when signal transmission occurs so that actuation occurs in advance of a signal deviation (such as minutes before a heart arrythmia or a hypoglycemic episode).

### Example 4: Microneedle Electrode for Transmitting Signals into THE Body

A microneedle made from stainless steel shim was produced with a 40° insertion angle, 0.4 mm base width, and 0.75 mm penetration depth (FIG. 1A, FIG. 4A). In some embodiments, these microneedles may have penetration depths of 0 mm, 0.25 mm, 0.3 mm, 0.6 mm, 0.75 mm, 0.9 mm, 1.25 mm. These microneedles are capable of penetrating through murine skin and passing signals from outside of the body into the dermis. Bypassing the highly resistive epidermis and stratum corneum prevents additional signal attenuation and ensures maximum voltage gradient generation across the body. In addition to using microneedles, it may also be possible to deliver a voltage pulse without needle penetration using a conductive gel or pre-made holes in the skin to reduce the impedance. Transmitting the signal across the highly resistive epidermis and stratum corneum with 0 mm penetration depth is also possible. The microneedles are connected to external electronics, such as printed circuit boards (PCBs) by using silver conductive paste. In some embodiments, the microneedles are connected to soft wearable electronics such as flexible printed circuit boards (FPCBs) or screen-printed elastomers via eutectic Gallium Indium (EGaIn). In some embodiments, the microneedles could be made of or be made with other metals (e.g., titanium), semiconductors (PEDOT: PSS), ceramics (silicon), and/or polymers (poly D, L-lactic-co-glycolic acid (PLGA) and poly-ethylene glycol (PEG)).

The microneedles can increase the generated voltage gradient through the body (FIG. 4C, FIG. 4D). A voltage gradient is still generated with microneedles that penetrate the skin at a depth of 0 mm.

### Example 5: Microneedle Spatial Orientation for Transmitting Signals to Specific Organs

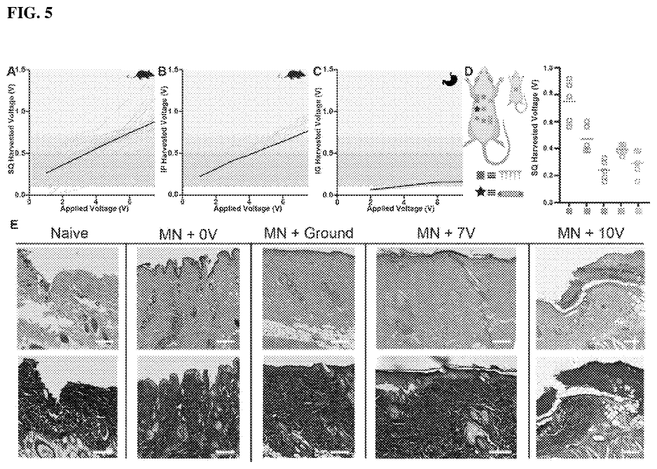

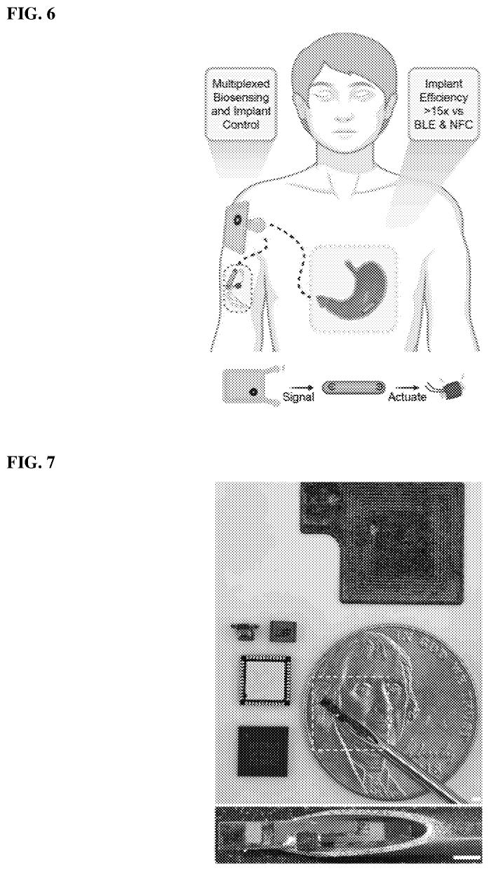

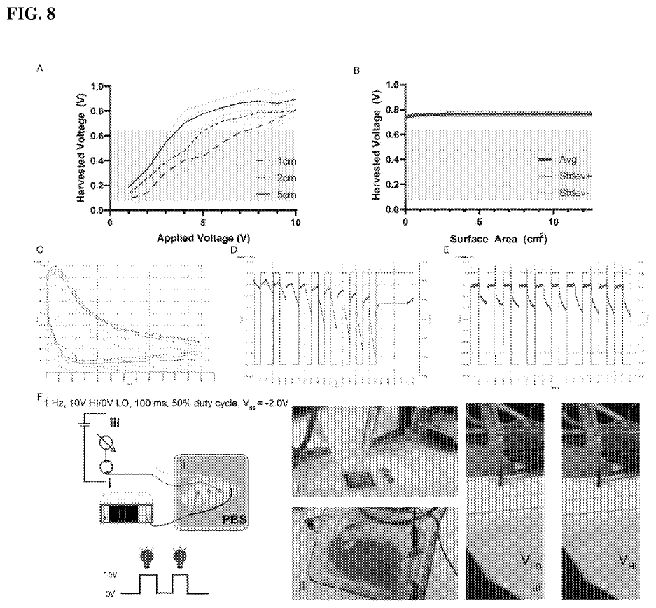

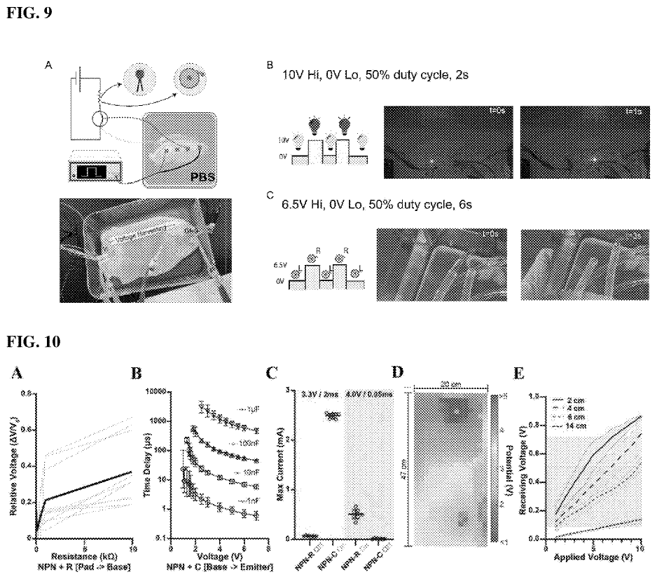

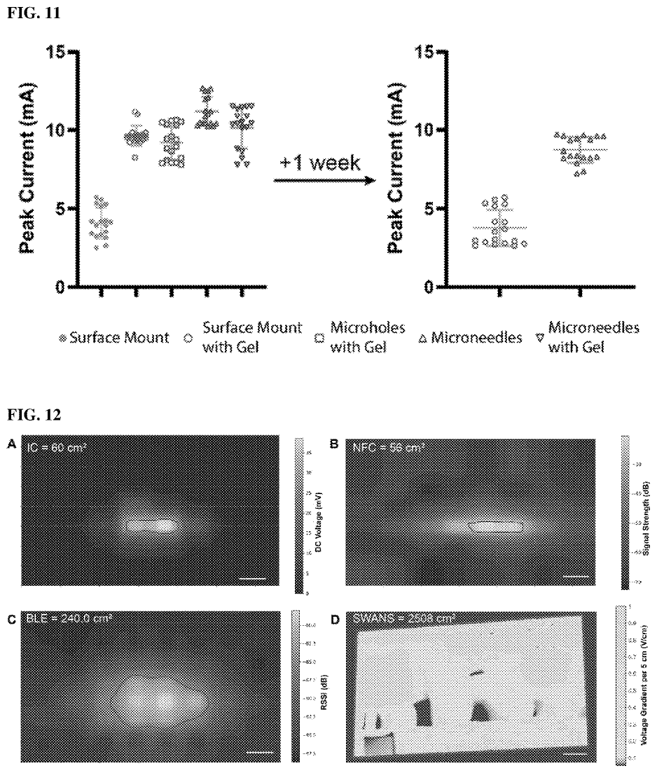

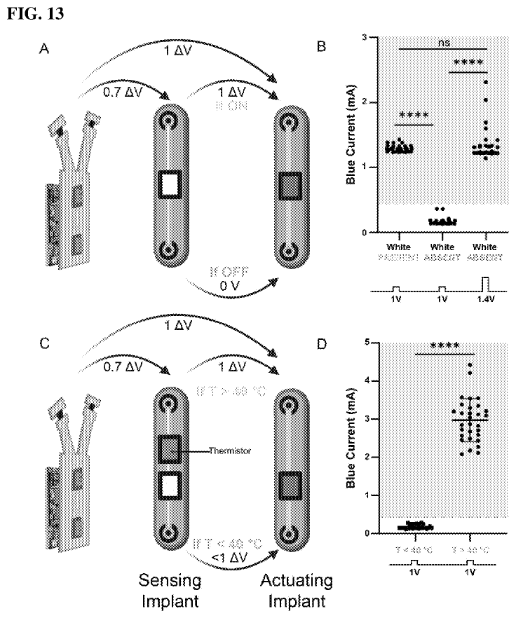

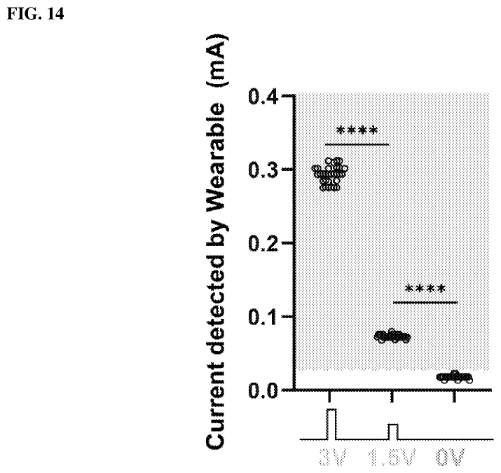

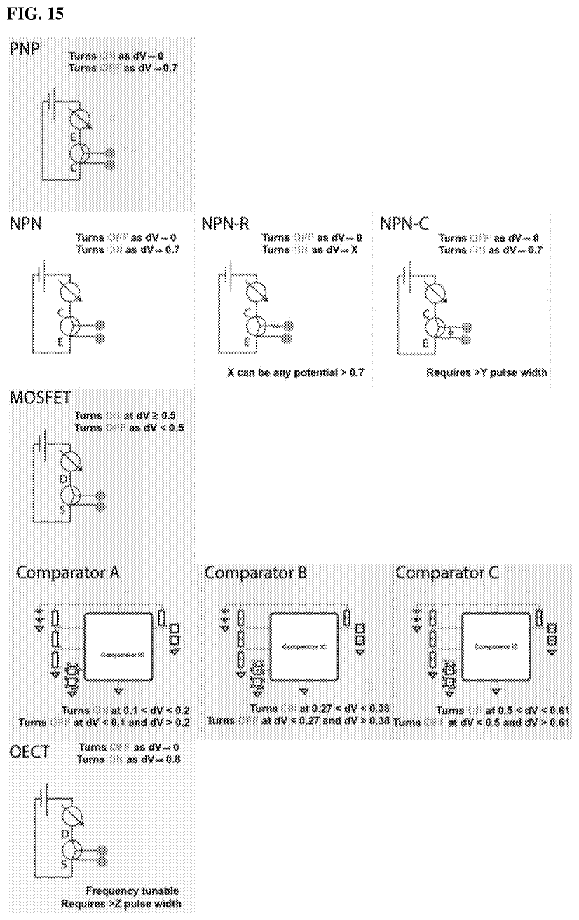

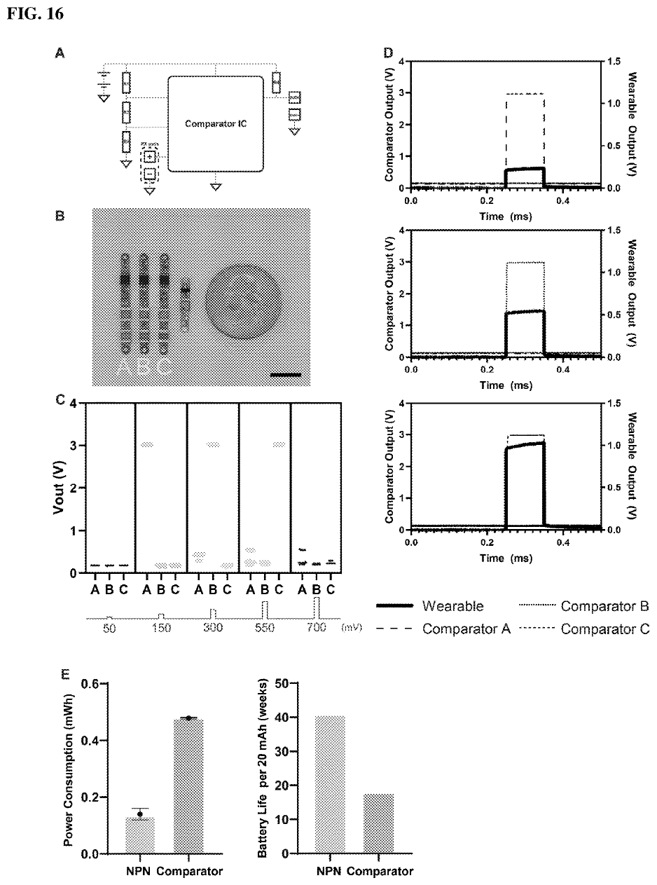

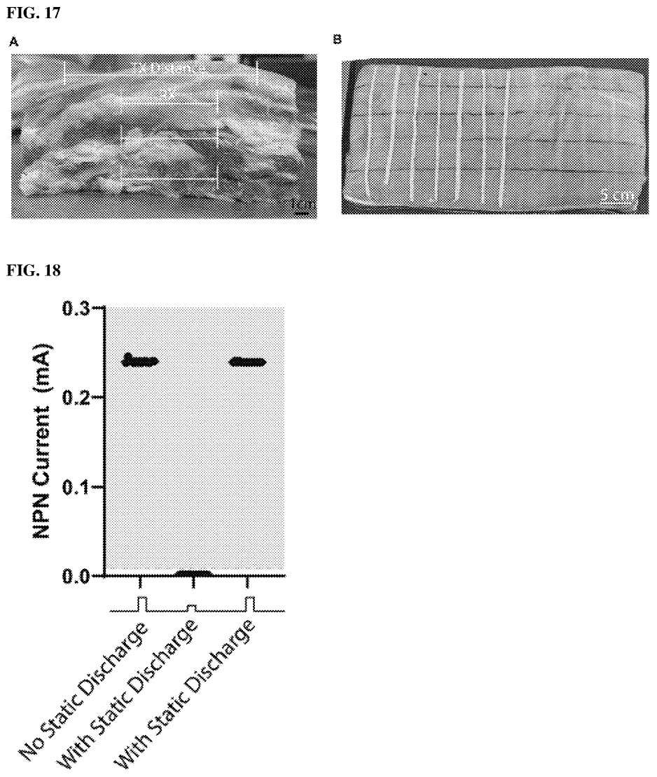

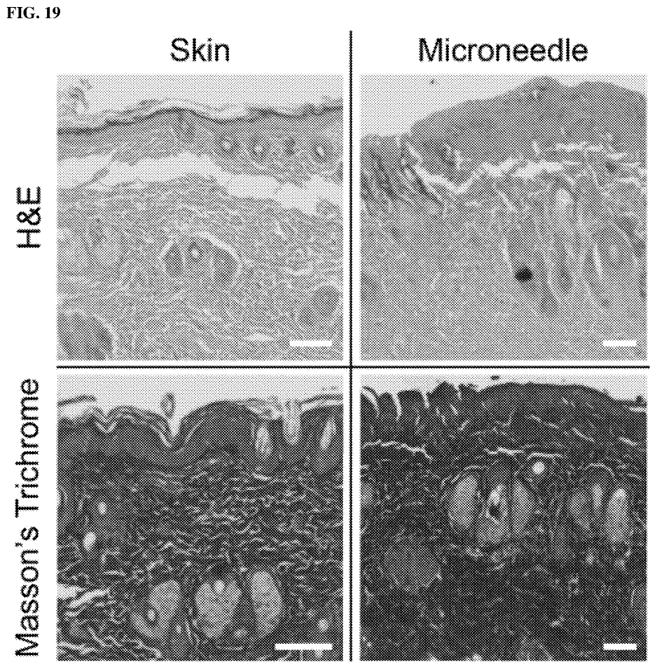

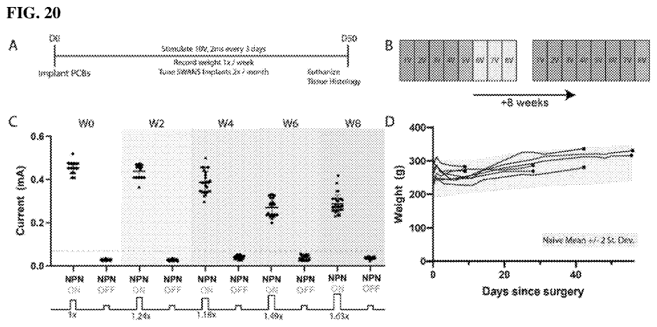

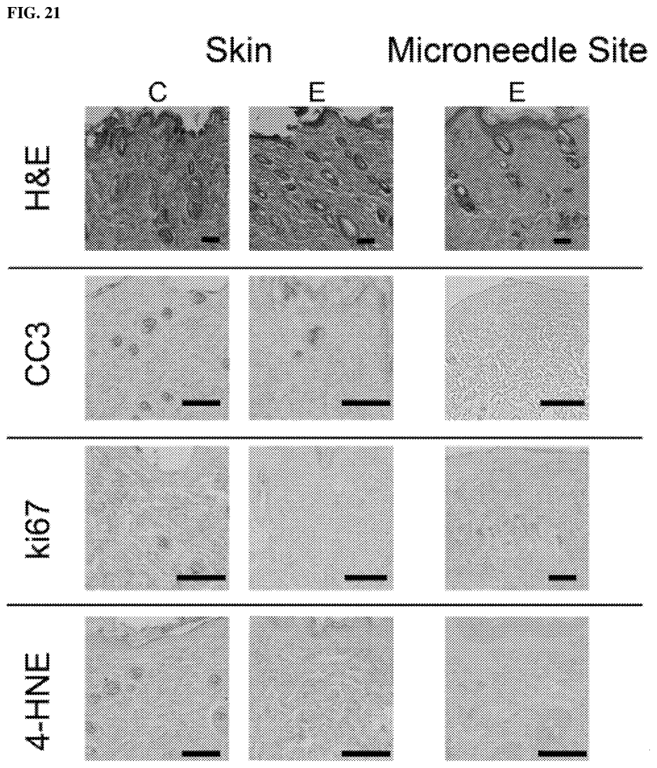

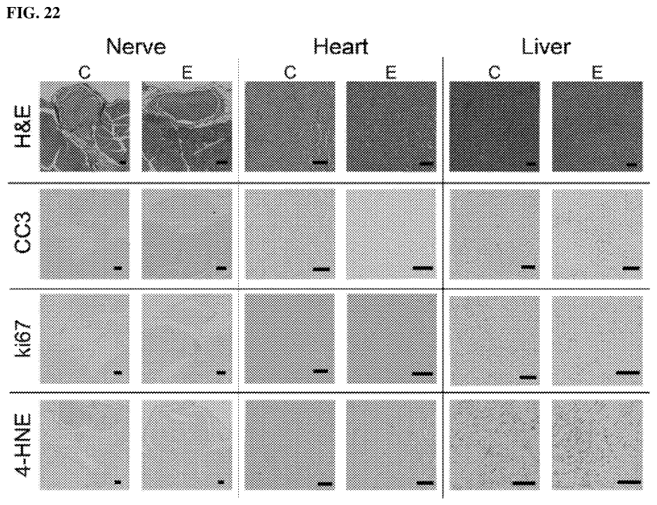

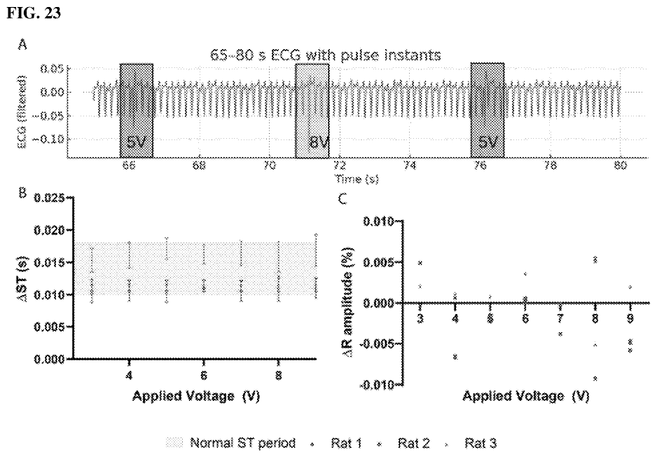

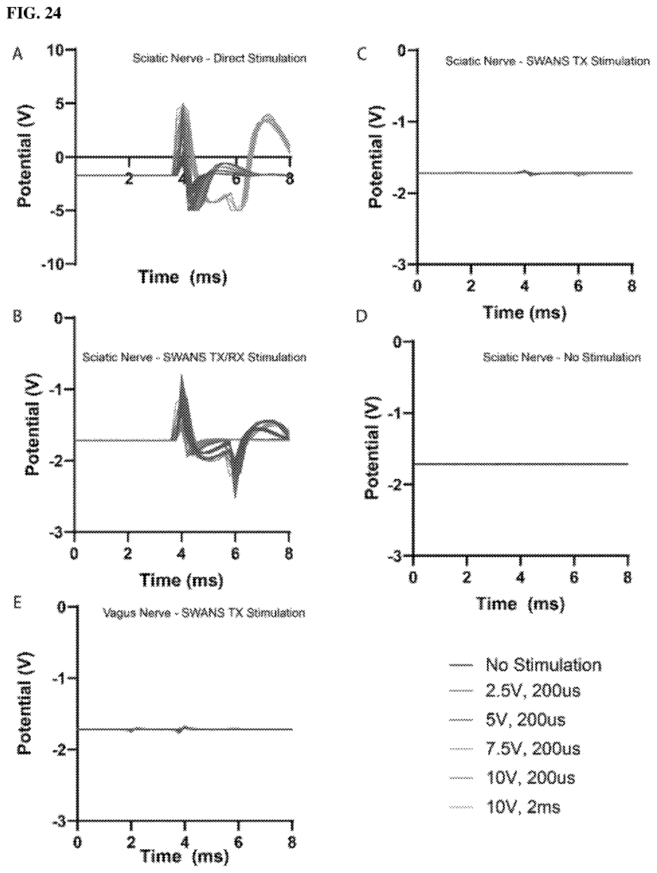

Because of the intimate connection between probe separation (FIG. 1C, FIG. 1E) and applied voltage (FIG. 1D), it is possible to change the location of the signal transmitting TX microneedles or patches to selectively turn on a device in a key region of tissue. Generated voltage gradients vary based on the location of the signal injecting transmitter (FIG. 5D). In some embodiments, when trying to communicate with a gastric-resident device or another device, the wearable transmitters may be placed on the belly and the back, or the belly and the belly, or on the back and the back. Wearable transmitters on other locations of the body could also work. In some embodiments, the wearable transmitter may be placed on the arm, thigh, scalp, or other on-body locations. The wearable transmitter may be placed close to the receiver or may be placed far on the receiver.

### Example 6: Thin Planar Electrode for Receiving Signals Inside the Body

Our communication system relies on 2 tissue-interfacing electrodes that continuously detect changes in electric fields throughout the body. These electrodes are fabricated by depositing polydimethylsiloxane (PDMS) on a glass substrate via spin-coating. The PDMS can be spin-coated at 300, 400, 500, 600, 700, 800, 900, or 1000 rpm for 1 minute to yield thicknesses ranging between 1000 μm and 25 μm. The PDMS has a layer of Chromium, and a layer of Gold deposited onto the surface making it electrically conductive. In some embodiments, an additional layer of chromium could be deposited onto the gold. The pads are conducted to external electronics, such as printed circuit boards (PCBs) by using eutectic gallium indium (EGaIn) or silver paste. Alternatively, receiving electrodes can be made directly on the PCB surface by depositing a layer of gold onto the polyimide substrate that the PCB is created from.

### Example 7: Signal Reception in the Subcutaneous Space, Intraperitoneal Space, or the Gastric Space

Our communication system allows voltage gradients to be observed in any portion of continuous electrolyte-soaked tissue. This means that devices could be triggered regardless of their location within the human body. The communication system does not require intimate knowledge of the current location of the internalized device which is important as subcutaneously implanted devices can migrate along with vaginal implants. Such an event would not disrupt our communication. A receiving device could be located in the subcutaneous space (FIG. 5A). A receiving device could be located in the intraperitoneal space (FIG. 5B). A receiving device could be located in the gastrointestinal tract (FIG. 5C). Irrespective of device location, the wearable is able to transmit a signal, and the internalized device is able to receive the signal.

### Example 8: Alternative Devices to Be Utilized With Signal Transmission

The transistor circuit can be integrated with a number of electromechanical devices. We have demonstrated use in light emitting diodes (LEDs), nerve cuffs, pumps, and triggerable auto-injectors. It can also be used in other electrical stimulators such as gastric stimulators or pacemakers. It can also be used with other electromechanical devices such as motors or solenoids. It can also be used with sensors. It can also be used with microcontrollers that have dedicated BLE modules.

### Example 9: Signal Transmission From Within the Body

The communication system enables signal transmission from an internalized device to another internalized device or to a wearable device. The internalized device can be interrogated by the wearable and consequently apply its own voltage gradient across the surrounding tissue. The wearable or another internalized device in the vicinity can detect this additional voltage gradient. The wearable or internalized device could be an actuator such as an LED, nerve cuff, pump, or triggerable auto-injector. It can also be a sensor. The signal transmission can be logic-gated and tied to a sensed modality such as temperature, chemical composition, pH, pressure, or other data.

### INCORPORATION BY REFERENCE

All U.S. and PCT patent application publications and U.S. patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1-90. (canceled)

91. A device, comprising:
a first electronic device in contact with tissue;
a second electronic device in contact with tissue; and
a harvesting pad(s);
wherein:
the first electronic device is placed inside or outside of the body;
the second electronic device is placed inside or outside of the body;
the first electronic device is configured to transmit an electrical signal through the tissue to the second electronic device, wherein a voltage gradient within the body is generated;
the harvesting pad(s) is/are configured to harvest the generated voltage gradient, wherein the harvesting pad is placed at a predetermined distance within the body; and
the harvesting pad(s) is/are connected to the second electronic device.

92. The device of claim 91, wherein:
the first electronic device is an implantable printed circuit board (PCB) and the second electronic device is a wearable printed circuit board (PCB), and the implantable PCB is configured to transmit an electrical signal through the tissue to the wearable PCB;
the first electronic device is a first wearable printed circuit board (PCB) and the second electronic device is a second wearable printed circuit board (PCB), and the first wearable PCB is configured to transmit an electrical signal through the tissue to the second wearable PCB;
the first electronic device is a first implantable printed circuit board (PCB) and the second electronic device is a second implantable printed circuit board (PCB), and the first implantable PCB is configured to transmit an electrical signal through the tissue to the second implantable PCB;
the first electronic device is an implantable printed circuit board (PCB) and the second electronic device is an ingestible printed circuit board (PCB), and the implantable PCB is configured to transmit an electrical signal through the tissue to the ingestible PCB;
the first electronic device is a first ingestible printed circuit board (PCB) and the second electronic device is a second ingestible printed circuit board (PCB), and the first ingestible PCB is configured to transmit an electrical signal through the tissue to the second ingestible PCB;
the first electronic device is an ingestible printed circuit board (PCB) and the second electronic device is a wearable printed circuit board (PCB), and the ingestible PCB is configured to transmit an electrical signal through the tissue to the wearable PCB; or
the first electronic device is a wearable printed circuit board (PCB) and the second electronic device is an ingestible printed circuit board (PCB), and the wearable PCB is configured to transmit an electrical signal through the tissue to the ingestible PCB.

93. The device of claim 91, comprising:
a wearable printed circuit board (PCB);
an implantable printed circuit board (PCB); and
a harvesting pad(s);
wherein:
the wearable PCB is configured to transmit an electrical signal to the implantable PCB through the tissue, wherein a voltage gradient within the body is generated; and
the harvesting pad(s) is/are configured to harvest the generated voltage gradient, wherein the harvesting pad is placed at a predetermined distance within the body;
the harvesting pad(s) is/are connected to the implantable printed circuit board (PCB).

94. The device of claim 93, wherein the implantable PCB further comprises one or more transistors, wherein the transistor(s) is/are switched on and off by the harvested voltage gradient.

95. The device of claim 94, wherein the transistor(s) measures no greater than 158 mm² in surface area.

96. The device of claim 94, wherein one or more of the transistors is/are an NPN transistor, and the harvesting pads are connected to the base and emitter pins of the NPN transistor.

97. The device of claim 96, wherein the NPN transistor is switched on by a harvested voltage gradient of approximately 0.7 V.

98. The device of claim 94, wherein one or more of the transistors is/are a PNP transistor, and the harvesting pads are connected to the base and collector pins of the PNP transistor.

99. The device of claim 98, wherein the PNP transistor is switched on by a harvested voltage gradient of approximately 0.1 V.

100. The device of claim 94, wherein one or more of the transistors is/are a MOSFET or FET transistor, and the harvesting pads are connected to the gate and source pins of the MOSFET or FET transistor.

101. The device of claim 94, wherein one or more of the transistor(s) is/are a P55NF06L MOSFET transistor, wherein the P55NF06L MOSFET transistor is switched on by a harvested voltage gradient of approximately 0.5 V.

102. The device of claim 94, wherein one or more of the transistor(s) is/are an organic electrochemical transistor (OECT), and the harvesting pads are connected to the gate and source pins of the OECT transistor.

103. The device of claim 102, wherein the OECT is switched on by a harvested voltage gradient of approximately 0.8 V.

104. The device of claim 94, wherein the transistor is utilized to switch on a device selected from the group consisting of a neurostimulator, a degradable metal film, a pump, a charger, a light emitting diode (LED), a triggerable auto-injector, a motor, a pneumatic inflater, an electrolyser, and a microcontroller.

105. The device of claim 91, wherein the cross-sectional area of the harvesting pad(s) is 1 mm² or less.

106. The device of claim 91 comprising two or more harvesting pads, wherein the harvesting pads are placed 5 cm apart or less to harvest a voltage gradient of 0.7 V or more; 3 cm apart or less to harvest a voltage gradient of 0.3 V or more; or 0.5 cm apart or less to harvest a voltage gradient of 0.1 V or more.

107. The device of claim 93, wherein the wearable PCB further comprises microneedles, microneedle pads, and/or microneedle patches, wherein microneedles, microneedle pads and/or microneedle patches are configured to apply the electrical signal below the epidermis.

108. The device of claim 107, wherein the microneedles can reach depths of approximately 0.9, 0.75, 0.6 or 0.25 mm below the epidermis.

109. The device of claim 107, wherein the microneedle pads provide a 7V minimum voltage pulse to generate a voltage gradient of 0.7 V or more; a 3.5V minimum voltage pulse to generate a voltage gradient of 0.5 V or more; or a 1V minimum voltage pulse to generate a voltage gradient of 0.1 V or more.

110. The device of claim 93, wherein the wearable printed circuit board further comprises a sensor connected to the wearable PCB, wherein the sensor is a chemical or physical sensor selected from a group consisting of a glucose sensor, a physiological analyte, a strain sensor, a pressure sensor, an EEG sensor, and an EKG sensor; and the wearable PCB emits the electrical signal when the sensor reaches a threshold value.
